(19) United States
(12) Patent Application Publication
(10) Pub. No.: US 2026/0367066 A1
(43) Pub. Date: Nov. 19, 2026

(54) DETECTION SYSTEM

(71) Applicant: Japan Display Inc., Tokyo (JP)

(72) Inventors: Hitoshi SAITO, Tokyo (JP); Tomohiro IDE, Tokyo (JP)

(21) Appl. No.: 19/673,047

(22) Filed: May 11, 2026

(30) Foreign Application Priority Data
May 13, 2025 (JP) 2025-080366

(51) Int. Cl. G01N 33/483; G01N 21/01

(52) U.S. Cl. CPC G01N 33/4833; G01N 21/01; G01N 2021/0112

(57) ABSTRACT

A detection system includes a detection device and an information processing device. The detection device produces an output obtained by detecting light from a light source transmitted through an object to be detected. The information processing device includes: a storage configured to store reference data including information indicating an increase rate of a colony area after culturing with respect to a predetermined area assumed when the biological entities having the predetermined area are cultured for a predetermined time; a circuit configured to periodically generate clocks for measuring a lapse of the predetermined time; and a setter configured to newly set a relation between the predetermined time and the number of the generated clocks based on a relation between the increase rate of an actual colony area obtained based on the output indicating a result of culturing of the biological entities and the increase rate indicated by the information.

### CROSS-REFERENCE TO RELATED APPLICATION

This application claims the benefit of priority from Japanese Patent Application No. 2025-080366 filed on May 13, 2025, the entire contents of which are incorporated herein by reference.

### BACKGROUND

### 1. Technical Field

What is disclosed herein relates to a detection system.

### 2. Description of the Related Art

Detection system are known that culture a sample containing biological entities to be cultured such as microorganisms in a culture vessel placed on a photosensor, capture images of colonies that are formed during a culture period, and determine the microorganisms forming the colonies from a scattered light pattern of the colonies (for example, Japanese Patent Application Laid-open Publication No. 2018-033430).

For some of the biological entities to be cultured, there is a known relation between the lapse of time during which the biological entities to be cultured are placed in an environment suitable for culturing and the expansion degree of the colonies formed over the lapse of that time. The lapse of the time is desired to be managed based on the expansion degree of the colonies of the biological entities to be cultured, using such a relation. However, no detection system can achieve such management.

For the foregoing reasons, there is a need for a detection system capable of managing the lapse of the time based on the expansion degree of colonies of the biological entities to be cultured.

### SUMMARY

According to an aspect, a detection system includes a detection device and an information processing device that is provided so as to be communicable with the detection device. The detection device includes a planar optical sensor, a light source, and an object placement table. The light source is provided so as to be capable of emitting light toward a detection surface of the planar optical sensor. The object placement table is interposed between the planar optical sensor and the light source and provided to allow an object to be detected to be placed thereon. The object to be detected is configured to allow a culture medium with biological entities to be formed thereon. The detection device is configured to produce an output obtained by detecting the light from the light source transmitted through the object to be detected using the planar optical sensor. The information processing device includes: a storage configured to store reference data including information indicating a rate of increase of a colony area after culturing with respect to a predetermined area that is assumed when the biological entities having the predetermined area are cultured in a predetermined environment for a predetermined time; a circuit configured to periodically generate clocks for measuring a lapse of the predetermined time; and a setter configured to newly set a relation between the predetermined time and the number of the generated clocks based on a relation between the rate of increase of an actual colony area obtained based on the output indicating a result of culturing of the biological entities having the predetermined area for the predetermined time measured based on the number of clocks in the predetermined environment and the rate of increase indicated by the information.

### BRIEF DESCRIPTION OF THE DRAWINGS

FIG. 1 is a schematic block diagram illustrating a main configuration of a detection system;

FIG. 2 is a perspective view illustrating a configuration example of a detection device;

FIG. 3 is a perspective view illustrating a state in which a top panel has been removed from FIG. 2;

FIG. 4 is a schematic view illustrating a functional configuration of the detection device;

FIG. 5 is a functional block diagram illustrating a main functional configuration of the detection device and a main functional configuration of an information processing device;

FIG. 6 is a functional block diagram illustrating communication between the detection device and the information processing device, and the function of the information processing device in more detail than FIG. 5;

FIG. 7 is an explanatory diagram schematically illustrating states of a placement substrate before and after measurement of an elapsed time performed in a time measurement process and display output contents of a display related to the measurement of the elapsed time;

FIG. 8 is a table schematically illustrating contents of reference data;

FIG. 9 is a table illustrating an example of a relation between time and clock before the measurement of the time measurement process, measurement results of the time measurement process, and the relation between time and clock after the measurement of the time measurement process;

FIG. 10 is a diagram illustrating an example of relations between directories and files before and after addition of information by an image analysis function;

FIG. 11 is a diagram illustrating a display example of a file to which information on a culture medium, information on a species of bacteria used as biological entities to be cultured, and other information are added;

FIG. 12 is a flowchart of the time measurement process; and

FIG. 13 is a flowchart of a post-measurement process.

### DETAILED DESCRIPTION

The following describes an embodiment of the present disclosure with reference to the drawings. What is disclosed herein is merely an example, and the present disclosure naturally encompasses appropriate modifications easily conceivable by those skilled in the art while maintaining the gist of the present invention. To further clarify the description, the drawings may schematically illustrate, for example, widths, thicknesses, and shapes of various parts as compared with actual aspects thereof. However, they are merely examples, and interpretation of the present disclosure is not limited thereto. The same element as that described with reference to an already mentioned drawing is denoted by the same reference numeral through the present specification and the drawings, and detailed description thereof may not be repeated where appropriate.

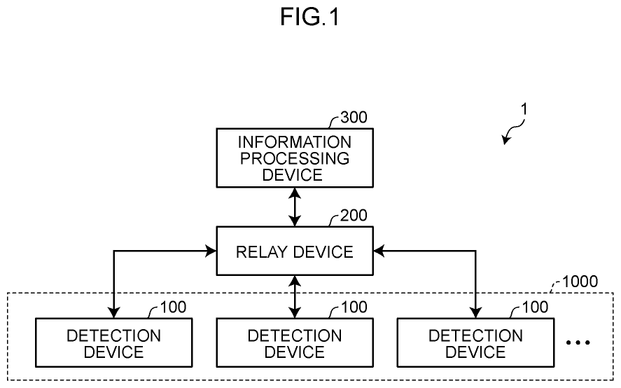

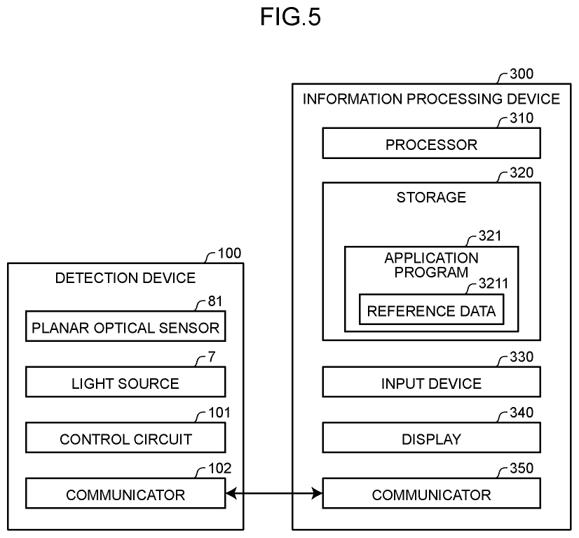

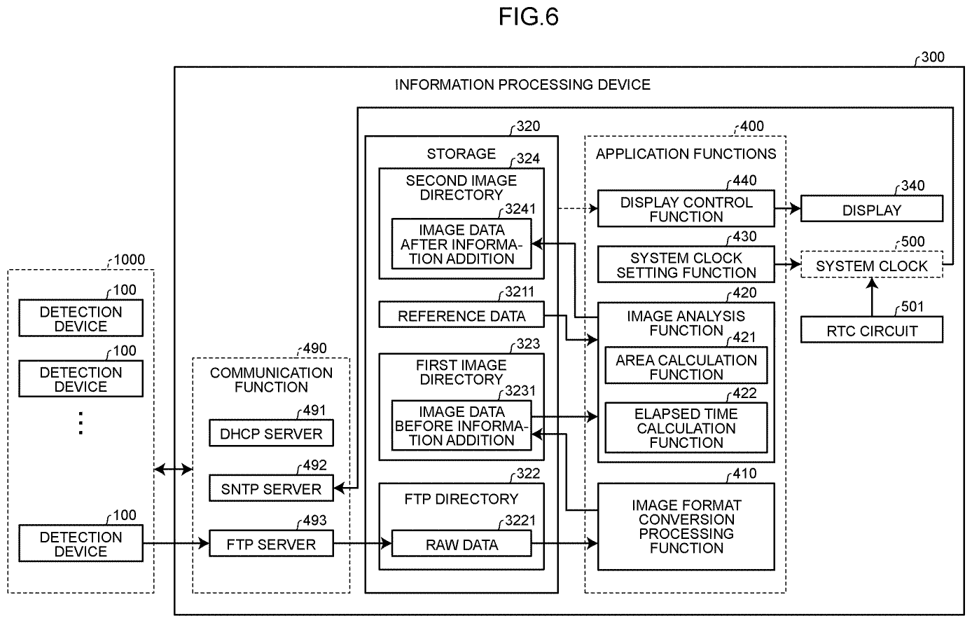

FIG. 1 is a schematic block diagram illustrating a main configuration of a detection system 1. The detection system 1 includes a detection device 100, a relay device 200, and an information processing device 300. In the description according to the embodiment, the information processing device 300 is coupled to a plurality of the detection devices 100 via the relay device 200, but the detection system 1 only needs to include one or more of the detection devices 100. The relay device 200 is not essential, and the detection device 100 may be directly coupled to the information processing device 300. As illustrated in FIG. 1, and FIGS. 5 and 6 to be explained later, the information processing device 300 of the embodiment is provided independently of the detection device 100.

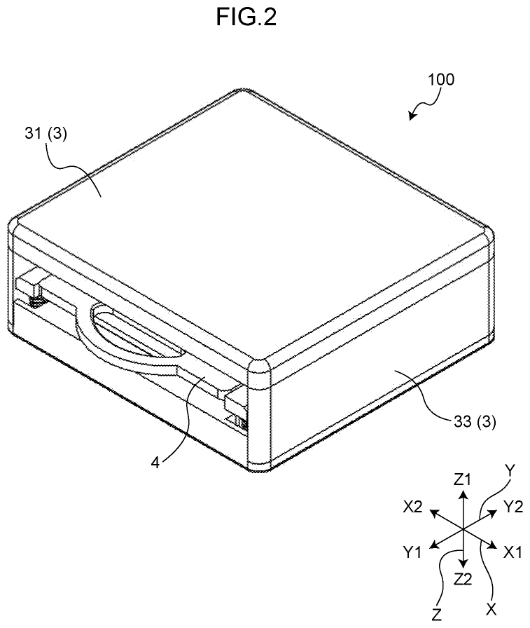

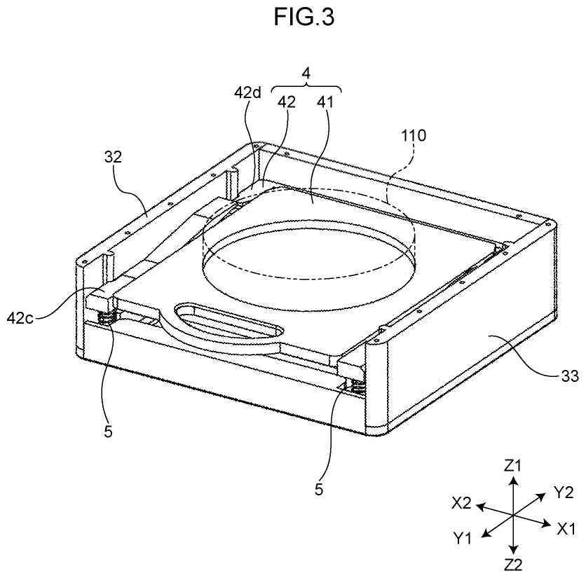

FIG. 2 is a perspective view illustrating a configuration example of the detection device 100. FIG. 3 is a perspective view illustrating a state in which a top panel has been removed from FIG. 2. In XYZ coordinates in FIGS. 2 to 4, a Z direction (first direction) corresponds to the up-down direction; an X direction (second direction) corresponds to the left-right direction; and a Y direction (second direction) corresponds to the front-rear direction. The X direction intersects (at right angles) the Y and Z directions; the Y direction intersects (at right angles) the X and Z directions; and the Z direction intersects (at right angles) the X and Y directions. A Z1 side is one side in the first direction, and a Z2 side is the other side in the first direction. In the present embodiment, the first direction is the Z direction, and the second direction is a direction that intersects the first direction. That is, the second direction is not limited to the X or Y direction, but the examples of the second direction include directions between the X and Y directions.

As illustrated in FIGS. 2 and 3, the detection device 100 has a substantially box shape. The detection device 100 includes a housing 3 and an object placement table 4. The housing 3 includes a top panel 31 and side panels 32 and 33. The object placement table 4 includes a plate 41 and a base plate 42. An object to be detected 110 is placed on the plate 41. Four corners of the base plate 42 are provided with a front holder 42c and a rear holder 42d. The front and rear holders 42c and 42d are urged upward (toward the Z1 side) by springs 5. Since the object to be detected 110 is placed on the plate 41, the plate 41 and the object to be detected 110 are urged upward (toward the Z1 side) by the springs 5.

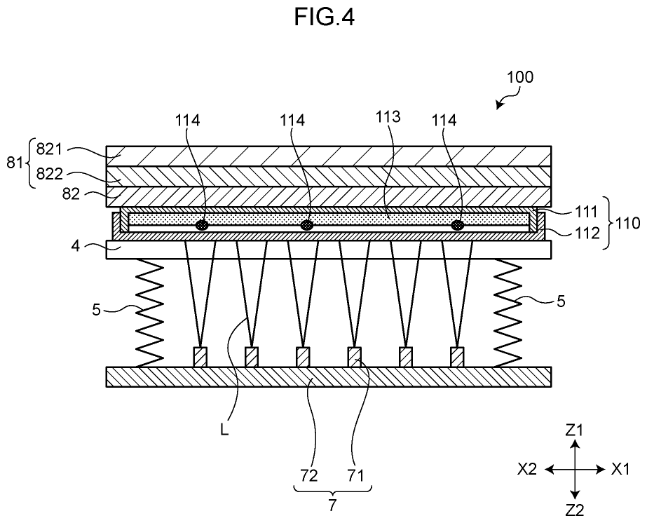

FIG. 4 is a schematic view illustrating a functional configuration of the detection device 100. As illustrated in FIG. 4, the detection device 100 includes a light source 7, the object to be detected 110, an optical filter 82, a planar optical sensor 81, and the springs 5.

The light source 7 includes a light source board 72 and a plurality of point light sources (light-emitting elements) 71. The point light sources 71 are light-emitting diodes (LEDs), for example. Thus, the light source 7 includes the point light sources 71 arranged in a planar configuration.

The object to be detected 110 includes a placement substrate 111 and a cover member 112. The object to be detected 110 is a Petri dish, for example. The object to be detected 110 has a light-transmitting property. The placement substrate 111 is a light-transmitting substrate that is disposed on the Z1 side of the light source 7 so as to overlap the light source 7, and on which a culture medium 113 (e.g., agar) is formed.

In the embodiment, the object to be detected 110 is placed upside down with respect to a normal container. That is, in the normal container, the placement substrate is located on the lower side and the cover member is located on the upper side. In contrast, in the object to be detected 110 according to the embodiment, the placement substrate 111 is located on the upper side while the cover member 112 is located on the lower side. In addition, the planar optical sensor 81 and the optical filter 82 are provided on the upper side (Z1 side) of the upside-down object to be detected 110 while the light source 7 is provided on the lower side (Z2 side) of the upside-down object to be detected 110. The culture medium 113 is provided on the lower side of the placement substrate 111. Colonies 114 can be formed on the culture medium 113 (surface on the lower side of the culture medium 113). The colonies 114 are colonies that are produced by culturing microorganisms such as bacteria, biological tissues, or the like (for example, biological entities to be cultured 115, which will be described later), and expand with the lapse of time under a predetermined environment to be described later.

The planar optical sensor 81 includes an array substrate 821 and a sensor pixel 822 (photodiode or photodetection element). The planar optical sensor 81 is located on the Z1 side with respect to the optical filter 82 so as to overlap the optical filter 82. A plurality of the sensor pixels 822 are provided on a surface on the Z2 side of the array substrate 821. The optical filter 82 is an optical element that transmits, toward the photodiodes, components of light L emitted from the point light sources 71 that travel in a direction orthogonal to the planar optical sensor 81. The optical filter 82 is also called collimating apertures or a collimator.

The light L emitted from the point light sources 71 passes through the cover member 112, the culture medium 113, the placement substrate 111, and the optical filter 82, and is received by the planar optical sensor 81. The amount of light emitted to the photodiode (photodetection element) of the planar optical sensor 81 differs between the following two regions: one of the two regions is a region where the microorganisms, such as the biological entities to be cultured 115 or the colonies 114 illustrated in FIG. 4, are significantly present; and the other of the two regions is a region where the microorganisms or the like are absent or extremely low in density even if present. This difference allows the planar optical sensor 81 to capture an image that indicates the region where the microorganism or the like are significantly present. Thus, the object to be detected 110 is placed between the light source 7 and the planar optical sensor 81, and the light from the light source 7 is detected with the planar optical sensor 81, whereby the detection device 100 enables monitoring of changes in microorganisms and the like in the object to be detected 110.

More specifically, the sensor pixels 822 are two-dimensionally arranged on a surface (light detection surface) of the planar optical sensor 81 on the light source 7 side. The photodiodes individually provided in the sensor pixels 822 individually detect the light, thereby obtaining two-dimensional outputs representing detection results of the light on the light detection surface. The term “image” refers to an image represented by the outputs of the detection device 100, which serves as a two-dimensional image reflecting the intensities of the light detected by the planar optical sensor 81 of the detection device 100, unless otherwise noted. The term “file” refers to data of the image, unless otherwise noted. The term “file name” refers to the name of the file.

As illustrated in FIG. 4, the detection device 100 includes the planar optical sensor (planar optical sensor 81), the light source (light source 7), and the object placement table (object placement table 4). The light source (light source 7) is provided so as to be capable of emitting the light toward the detection surface of the planar optical sensor. The object placement table (object placement table 4) is interposed between the planar optical sensor and the light source and provided to allow the object to be detected (object to be detected 110) to be placed thereon. The object to be detected is configured to allow the culture medium (culture medium 113) to be formed thereon. The detection device 100 produces the output obtained by detecting the light from the light source transmitted through the object to be detected using the planar optical sensor. As will be described later, the culture medium can be provided with the biological entities to be cultured (the biological entities to be cultured 115). The colonies (colonies 114) can be formed by culturing the biological entities to be cultured (hereinafter simply referred to as “biological entities”).

The relay device 200 illustrated in FIG. 1 relays the signals and/or power supply between the detection device 100 and the information processing device 300. Specifically, the detection device 100 in the embodiment has a Universal Serial Bus (USB) port for receiving power supply and a Local Area Network (LAN) port for communicating with the information processing device 300. The relay device 200 includes a USB hub and a LAN hub (for example, switching hub). In the embodiment, the detection devices 100 operate by receiving the power supply via the USB hub included in the relay device 200. In the embodiment, the detection devices 100 are communicably coupled to the information processing device 300 via the relay device 200.

The LAN hub of the relay device 200 is coupled to the LAN port included in the information processing device 300, thereby relaying signals between the detection device 100 and the information processing device 300. The USB hub of the relay device 200 may be configured to obtain power supplied to the detection device 100 by being coupled to the USB port included in the information processing device 300, or configured to obtain power supplied to the detection device 100 by being coupled to an independent power supply. If the detection device 100 is directly coupled to the information processing device 300 without the relay device 200, the LAN port included in the detection device 100 is coupled to the LAN port included in the information processing device 300 via a LAN cable. In this case, the USB port included in the detection device 100 is coupled to the USB port included in the information processing device 300 via a USB cable. A configuration in which both power supply and transmission/reception of data are performed through USB coupling may be employed for the coupling between the detection device 100 and the information processing device 300.

The relay device 200 of the embodiment is merely a configuration that supplies the power to the detection device 100 and relays the communication between the detection device 100 and the information processing device 300. That is, the relay device 200 does not make any substantive changes to the content of the communication between the detection device 100 and the information processing device 300.

FIG. 5 is a functional block diagram illustrating a main functional configuration of the detection device 100 and a main functional configuration of the information processing device 300. In FIG. 5 and FIG. 6 to be explained later, the relay device 200, which does not make any substantive changes to the content of the communication between the detection device 100 and the information processing device 300, is not illustrated.

The detection device 100 further includes a control circuit 101 and a communicator 102, in addition to the planar optical sensor 81 and the light source 7 described with reference to FIG. 4. The control circuit 101 controls operations of the planar optical sensor 81, the light source 7, and the communicator 102. Thus, the control circuit 101 performs the operation control of the light source 7 and the planar optical sensor 81 related to the operation to detect the light emitted from the light source 7 using the planar optical sensor 81. The communicator 102 includes, in the configuration thereof, a circuits that performs processing related to communication with the information processing device 300. The LAN port included in the detection device 100 is provided in the communicator 102. The output of the planar optical sensor 81 obtained correspondingly to the intensity of the light detected by the planar optical sensor 81 is transferred as raw data to the information processing device 300 via the communicator 102 under the control of the control circuit 101.

The information processing device 300 include a processor 310, a storage 320, an input device 330, a display 340, and a communicator 350. Although not illustrated in FIG. 5, the information processing device 300 further includes a real-time clock (RTC) circuit 501, as illustrated in FIG. 6 to be explained later.

The processor 310 includes, in the configuration thereof, a central processing unit (CPU) including an arithmetic circuit. The processor 310 performs what is called software processing to perform various functions by executing computer programs and other information. In the embodiment, the term “computer programs and other information” herein refers, for example, to “software programs and data referenced in execution of the software programs” stored in the storage 320, but is not limited thereto. For example, computer programs and other information that can be accessed externally through communication via the communicator 350 may be further used.

The storage 320 stores therein the computer programs and other information to be read out by the processor 310. FIG. 5 illustrates application programs 321 as the computer programs and other information. The application programs 321 are what are called the software programs. The application programs 321 include reference data 3211 as data referenced in the execution of the application programs 321.

Although not illustrated in FIG. 5, the storage 320 further stores therein computer programs and other information, as an operating system (OS) to be executed by the processor 310. The storage 320 includes, in the configuration thereof, one or more storage devices. Examples of such storage devices include, but are not limited to, hard disk drives (HDDs), solid-state drives (SSDs), and flash memories. The arithmetic unit 310 includes, in the configuration thereof, at least one or more of these storage devices.

The input device 330 receives input operations for the information processing device 300. The input device 330 is configured with at least one or more input devices, such as a keyboard and a mouse. The input device 330 receives the input operations for the information processing device 300 by being operated by a human who is an administrator of the information processing device 300. Hereinafter, the term simply called “administrator” refers to the human who is the administrator of the information processing device 300.

The display 340 performs display output corresponding to the processing content of the information processing device 300. The display 340 includes one or more display devices, such as liquid crystal displays. The display 340 performs the display output under the control of the processor 310.

The communicator 350 includes, in the configuration thereof, a circuit related to communication with the detection device 100. The communicators 102 and 350 serve as network interface controllers (NICs). The communicators 102 and 350 communicate with each other using a predetermined protocol (such as the Internet Protocol). This establishes communication between the detection device 100 and the information processing device 300.

FIG. 6 is a functional block diagram illustrating the communication between the detection device 100 and the information processing device 300, and the function of the information processing device 300 in more detail than FIG. 5. As illustrated in FIG. 6, the information processing device 300 operates so as to perform a communication function 490 as a function related to the communication with the detection device 100. Computer programs and other information to serve as the communication function 490 may be included in the application programs 321, or in known computer programs and other information, such as the OS or other software (not illustrated). The processor 310 reads out and executes the computer programs and other information, and whereby, the information processing device 300 operates to perform the communication function 490.

The communication function 490 includes a function as a Dynamic Host Configuration Protocol (DHCP) server 491, a function as a Simple Network Time Protocol (SNTP) server 492, and a function as a File Transfer Protocol (FTP) server 493.

The DHCP server 491 assigns an individual Internet Protocol (IP) address to each of the detection devices 100 based on the Dynamic Host Configuration Protocol (DHCP).

The SNTP server 492 provides information indicating the current time to the detection device 100 through the communication based on the Simple Network Time Protocol (SNTP). The current time herein includes information indicating a year, a month, a day, an hour, a minute and a second. The control circuit 101 of the detection device 100 manages the lapse of time based on the information given by the SNTP server 492.

The FTP server 493 performs file transfer between the detection device 100 and the information processing device 300 based on the File Transfer Protocol (FTP). The term “file” herein refers, for example, to the raw data mentioned above, but other data may also be subject to the file transfer by the FTP server 493. FIG. 6 illustrates the raw data transferred from the detection device 100 using the function of the FTP server 493 as raw data 3221. The information processing device 300 holds the raw data 3221 by storing the raw data 3221 in the storage 320.

As illustrated in FIG. 6, an FTP directory 322, a first image directory 323, and a second image directory 324 are set in the storage 320 of the embodiment. The FTP directory 322 is a directory where the raw data 3221 is stored. The first image directory 323 is a directory where image data 3231 before information addition is stored. The second image directory 324 is a directory where image data 3241 after information addition is stored. Hereinafter, the image data 3231 before information addition is referred to as a pre-information-addition image data 3231, and the image data 3241 after information addition is referred to as a post-information-addition image data 3241. The pre-information-addition image data 3231 and the post-information-addition image data 3241 will be described later.

As illustrated in FIG. 6, the various functions included in the communication function 490 are provided to all the detection devices 100. The file transfer by the FTP server 493 is individually performed between each of the detection devices 100 and the information processing device 300.

As illustrated in FIG. 6, the information processing device 300 operates to execute application functions 400. In the embodiment, the computer programs and other information to serve as the application functions 400 are the application programs 321. The processor 310 reads out and executes the application programs 321, and whereby, the information processing device 300 operates to execute the application functions 400.

The application functions 400 include an image format conversion processing function 410, an image analysis function 420, a system clock setting function 430, and a display control function 440.

The image format conversion processing function 410 is a function to convert the raw data 3221 into image data in a predetermined image format. Specifically, the image format conversion processing function 410 reads out the raw data 3221 from the FTP directory 322, converts the read out raw data 3221 into an image in the predetermined format, and writes the image into the pre-information-addition image data 3231. That is, the pre-information-addition image data 3231 is a file obtained by converting the raw data 3221 into the image data in the predetermined image format.

In the embodiment, Portable Network Graphics (PNG) is employed as the predetermined image format, but this is only an example and the predetermined image format is not limited to this format. The predetermined format may be another image format, such as the Joint Photographic Experts Group (JPEG).

The image analysis function 420 includes an area calculation function 421 and an elapsed time calculation function 422. The area calculation function 421 is a function to calculate the area of the cultured biological entities 115 in an image of the placement substrate 111 included in the pre-information-addition image data 3231. The elapsed time calculation function 422 is a function to calculate the elapsed time of a time measurement process based on the area of the biological entities 115 calculated by the area calculation function 421. Details of the area calculation function 421, the elapsed time calculation function 422, and the time measurement process will be described later.

Although not illustrated in FIG. 6, the image analysis function 420 may further include a function to identify the type of the biological entities 115 being cultured on the placement substrate 111 based on the image of the biological entities 115 included in the image of the placement substrate 111 included in the pre-information-addition image data 3231. The function to identify the type of the biological entities 115 may be performed based on known pattern matching or the like, and thus is not particularly noted in the present disclosure.

The system clock setting function 430 sets a system clock 500 based on the elapsed time of the time measurement process calculated by the elapsed time calculation function 422. The display control function 440 controls the display output contents by the display 340. Details of the system clock setting function 430, the display control function 440, and the system clock 500 will be described below.

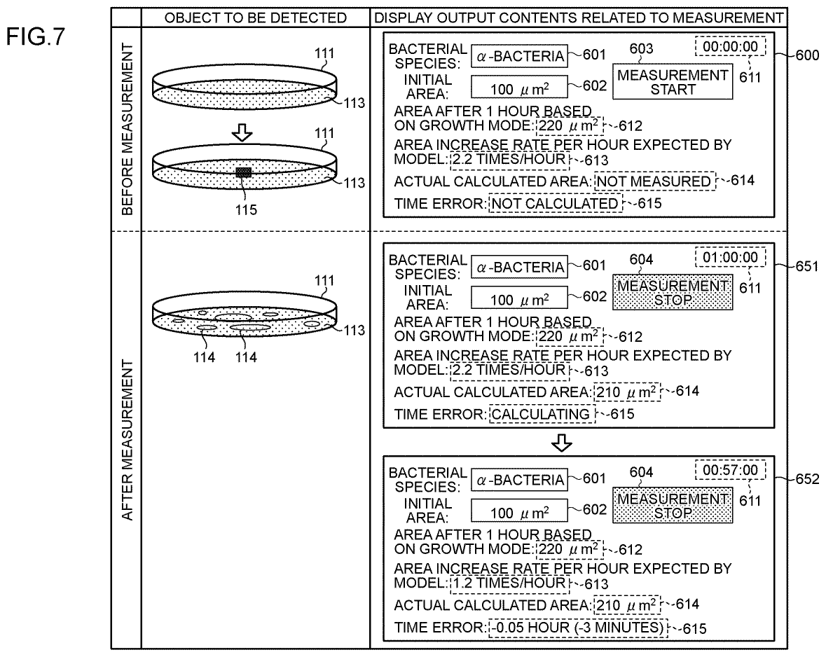

The following describes more details of the image analysis function 420, the system clock setting function 430, the display control function 440, and matters related to these functions, among the functions included in the application functions 400. First, the image analysis function 420 and matters related to the image analysis function 420 (time measurement process) will be described with reference to FIG. 7.

FIG. 7 is an explanatory diagram schematically illustrating states of the placement substrate 111 before and after the measurement of the elapsed time performed in the time measurement process and the display output contents of the display 340 related to the measurement of the elapsed time.

The time measurement process is performed for the purpose of measuring the elapsed time of a culturing process of the biological entities 115 associated with an expansion of the area of the colonies 114 of the biological entities 115 (refer to FIG. 7) that are cultured in the culture medium 113, based on the degree of the expansion of the area of the colonies 114. The time measurement process includes a pre-measurement process, a mid-measurement process, and a post-measurement process. The pre-measurement process includes two processes. One of the two processes is a process of applying (inoculating) the biological entities 115 onto the culture medium 113. The other one of the two processes is a process of: setting, in the detection device 100, the object to be detected 110 including the placement substrate 111 with the biological entities 115 inoculated onto the culture medium 113; and starting the measurement. The mid-measurement process is the culturing process of the biological entities 115 after the start of the measurement. The post-measurement process is a calculation process after the culturing process of the biological entities 115. The following sequentially describes these processes.

First, in the time measurement process, the process of applying (inoculate) the biological entities 115 onto the culture medium 113 is performed as a process included in the pre-measurement process. In FIG. 7, the placement substrate 111 before the biological entities 115 are applied onto the culture medium 113 and the placement substrate 111 after the biological entities 115 are applied onto the culture medium 113 are illustrated so as to be arranged vertically in an “object to be detected” column of a “before measurement” row. That is, in the process of applying the biological entities 115 onto the culture medium 113, the biological entities 115 are applied onto the culture medium 113. Hereinafter, the term “measurement target” refers to the object to be detected 110 including the placement substrate 111 with the biological entities 115 inoculated onto the culture medium 113.

The type of the biological entities 115 to be applied onto the culture medium 113 is determined in advance. FIG. 7 uses the expression “α-bacteria” as a title to indicate the type of the biological entities 115. The α-bacteria are Escherichia coli, for example.

The biological entities 115 subjected to the time measurement process in the embodiment are microorganisms that can be cultured on the culture medium 113, but the biological entities 115 are not limited thereto. The biological entities 115 may be, for example, biological tissues that can be cultured on the culture medium 113.

In the time measurement process, the process of: setting, in the detection device 100, the object to be detected 110 including the placement substrate 111 with the biological entities 115 inoculated onto the culture medium 113; and starting the measurement is performed as the process included in the pre-measurement process. Specifically, “setting, in the detection device 100, the object to be detected 110 including the placement substrate 111 with the biological entities 115 inoculated onto the culture medium 113” refers to “placing the measurement target on the plate 41 of the detection device 100”. In addition, “starting the measurement” refers to “pressing a measurement start button 603 on an initial setting screen 600 while the detection device 100 with the measurement target placed on the plate 41 is in an environment suitable for culturing the biological entities 115 in the measurement target”. The detection device 100 of the embodiment, with the object to be detected 110 set therein, is placed in an incubator. The incubator herein is a container-like device that maintains constant environmental conditions such as temperature and humidity inside. In the embodiment, the incubator is illustrated with dashed lines as an incubator 1000 (refer to FIG. 1).

The incubator 1000 is not essential, and the detection device 100 only needs to be placed under conditions where an environment suitable for growing the biological entities 115 is maintained. For example, the detection device 100 may be provided in a room that can maintain an environment suitable for growing the biological entities.

In the time measurement process, the initial setting screen 600 is displayed on the display 340. The process for displaying the initial setting screen 600 is performed by the display control function 440 described above. A “display output contents related to measurement” column of the “before measurement” row in FIG. 7 illustrates the initial setting screen 600. The initial setting screen 600 includes a bacterial species setting field 601, an initial area setting field 602, the measurement start button 603, an elapsed time display field 611, an assumed area display field 612, an area increase rate display field 613, a measured area display field 614, and a measurement result display field 615.

The bacterial species setting field 601 is a field that indicates the type of the biological entities 115 applied onto the culture medium 113. The initial area setting field 602 is a field that indicates an initial area of the biological entities 115 applied onto the culture medium 113. The specific contents of the bacterial species setting field 601 and the initial area setting field 602 are manually set by the administrator.

The measurement start button 603 is a button for entering a trigger to make a transition from the pre-measurement process to the mid-measurement process, to the information processing device 300. An operation of the measurement start button 603 is performed by the administrator. That is, when the measurement start button 603 is operated, the pre-measurement process is completed, and the process transitions to the mid-measurement process. Therefore, it is assumed that, at the time when the measurement start button 603 is operated, the measurement target is already placed on the plate 41 of the detection device 100 in the incubator and that the environment in the incubator is ready to be made suitable for culturing the biological entities 115. As the order of operation in the pre-measurement process, the input of the bacterial species setting field 601 and the initial area setting field 602 is preferably completed first. Then, the measurement target is preferably set in the detection device 100 in the incubator where an environment suitable for culturing the biological entities 115 is established, and the measurement start button 603 is preferably operated promptly thereafter.

The elapsed time display field 611 is a field where the elapsed time from when the measurement start button 603 is operated to when a measurement stop button 604 is operated is displayed.

In the embodiment, when the measurement start button 603 is operated, the measurement process transitions from the pre-measurement process to the mid-measurement process, and the measurement start button 603 is replaced with the measurement stop button 604. The measurement stop button 604 is a button for entering a trigger to make a transition from the mid-measurement process to the post-measurement process, to the information processing device 300. An operation of the measurement stop button 604 is performed by the administrator. That is, when the measurement stop button 604 is operated, the mid-measurement process is completed, and the process transitions to the post-measurement process. The elapsed time displayed in the elapsed time display field 611 during the mid-measurement process is the elapsed time (pre-measurement time) measured by the information processing device 300 using the system clock 500 at that time.

The assumed area display field 612 is a field that indicates an area of assumed colonies (assumed area) if the elapsed time from when the mid-measurement process starts is a predetermined time. The term “assumed colonies” herein refers to the colonies 114 that are assumed to be formed on the culture medium 113 of the measurement target as a result of the culturing of biological entities 115. The specific contents of the assumed area display field 612 correspond to the combination of the bacterial species setting field 601 and the initial area setting field 602. The specific contents of the assumed area display field 612 may be set and entered by the administrator, but, in the embodiment, are automatically determined with reference to the reference data 3211. The predetermined time is, for example, one hour, but is not limited thereto, and can be changed as appropriate as long as the duration of the execution of the mid-measurement process is sufficient for significant formation and expansion of the colonies.

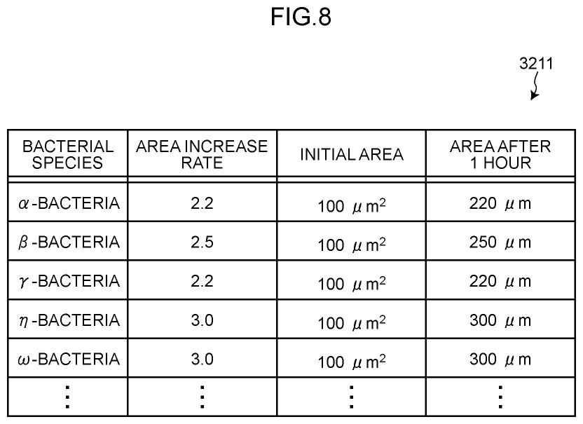

FIG. 8 is a table schematically illustrating the contents of the reference data 3211. The reference data 3211 is data in which a “bacterial species”, an “area increase rate”, an “initial area”, and an “area after one hour” are associated with each other.

The “bacterial species” is a character string indicating the type of the biological entities 115 applied (inoculated) onto the culture medium 113.

The “area increase rate” is a numerical value that indicates a multiple of the area of the “assumed colonies” relative to the initial area, which is assumed if the time during which the mid-measurement process is performed is the predetermined time. The “initial area” herein refers to the initial area of the biological entities 115 applied onto the culture medium 113, and corresponds to the contents displayed in the initial area setting field 602.

The “initial area” is a numerical value indicating the initial area of the biological entities 115 applied onto the culture medium 113. The “area after one hour” is a numerical value indicating the assumed area. The “assumed area” herein refers to the area of the colonies if the time during which the mid-measurement process is performed is the predetermined time. That is, the assumed area is a numerical value obtained by multiplying the “initial area” of a certain “bacterial species” by the “area increase rate” of the certain species. In FIG. 8, the column for the numerical value indicating the assumed area is titled as “area after one hour” assuming that the predetermined time is one hour, but if the predetermined time is other than one hour, the name “area after one hour” is preferably a name that indicates a time other than one hour.

In FIG. 8, α-bacteria, β-bacteria, γ-bacteria, η-bacteria, and ω-bacteria are listed as the “bacterial species”. The α-bacteria are Escherichia coli, for example. The β-bacteria are Salmonella, for example. The γ-bacteria are Listeria monocytogenes, for example. The η-bacteria are Streptococcus, for example. The ω-bacteria are Staphylococcus, for example. These species are only examples and not limited thereto. The bacterial species employed as the “bacterial species” may be other species for which formation patterns of colonies corresponding to culture time are already known.

In FIG. 8, the numerical value of the “area increase rate” is “2.2” for the α-bacteria and the γ-bacteria, “2.5” for the β-bacteria, and “3.0” for the η-bacteria and the ω-bacteria. These specific numerical values are set in advance based on the knowledge about formation patterns of colonies corresponding to the culture time. In FIG. 8, “100”μm² is uniformly set as values of the “initial area”. These values are set in advance correspondingly to the size of a standard specimen available as the biological entities to be cultured 115.

The predetermined environment of the culture medium 113, in which the correspondence between “bacterial species”, “initial area”, and “colony increase rate” indicated by the reference data 3211 is established, is determined in advance. To give a specific example, in the embodiment, the temperature and humidity of the culture medium 113 are maintained using the incubator 1000. More specifically, in the embodiment, the temperature (atmospheric temperature) and the humidity around the detection device 100 are conditions of the predetermined environment of the culture medium 113. The temperature (atmospheric temperature) is assumed to be in the range of 33 °C to 38 °C. The humidity is assumed to be a humidity at which no condensation occurs at the temperature in the range of 33 °C to 38 °C. When the temperature around the detection device 100 is in the range of 33 °C to 38 °C and the humidity around the detection device 100 is the humidity at which no condensation occurs at the temperature in the range of 33 °C to 38 °C, the temperature and humidity as the predetermined environment of the culture medium 113 can be maintained.

Thus, the storage 320 of the information processing device 300 stores therein the reference data (reference data 3211) including the rate (for example, 2.2) of increase of a colony area of the biological entities (for example, Escherichia coli) relative to a predetermined area (for example, 100 μm²), wherein the predetermined area is an initial area of the biological entities, and the colony area is an assumed area of the biological entities after the biological entities having the predetermined area are cultured in the predetermined environment for a predetermined time (for example, one hour).

In the embodiment, it is assumed that the reference data 3211 is also referenced for the setting of the bacterial species setting field 601 and the initial area setting field 602 (refer to FIG. 7) by the administrator that is performed in the pre-measurement process. Specifically, the bacterial species setting field 601 is provided, for example, as a pull-down selection item, so as to allow selection of any one of character strings for the “bacterial species” in the reference data 3211. If a character string indicating any one of the “bacterial species” is selected in the bacterial species setting field 601 by the administrator, the “initial area” is automatically set in the initial area setting field 602. The “initial area” set in the initial area setting field 602 is associated with the selected “bacterial species” in the reference data 3211. In the embodiment, in this case, the “area after one hour” associated with the character string indicating the selected “bacterial species” in the reference data 3211 is automatically set in the assumed area display field 612 (refer to FIG. 7). Furthermore, in the embodiment, in this case, the “area increase rate” that is associated with the character string indicating the selected “bacteria species” in the reference data 3211 is automatically set in the area increase rate display field 613 (refer to FIG. 7). The area increase rate display field 613 is a field that indicates the “area increase rate” if the time during which the mid-measurement process is performed is the predetermined time.

The input by the manual setting by the administrator is made via the input device 330. Thus, the input device 330 receives input to specify any one of the biological entities as a plurality of types of bacterial species illustrated, for example, in FIG. 8. As illustrated as the initial setting screen 600 in FIG. 7, the display 340 of the embodiment displays a screen that includes an entry field (bacterial species setting field 601) for specifying the biological entities.

The measured area display field 614 illustrated in FIG. 7 is a field that indicates the area of the “actual colonies” at the time of the transition to the post-measurement process, that is, at a completion time point of the mid-measurement process. The “actual colonies” herein refers to the colonies 114 formed on the culture medium 113 of the measurement target as a result of the culturing of the biological entities 115 during the elapsed time when the mid-measurement process is executed.

At the time of the pre-measurement process, the mid-measurement process has not yet been executed. Thus, at this time, the content of the measured area display field 614 on the initial setting screen 600 is “not measured” as illustrated in the “display output contents related to measurement” column of the “before measurement” row in FIG. 7. In contrast, the content of the measured area display field 614 on a post-measurement screen 651 is “210 μm²” as illustrated in the “display output contents related to measurement” column of an “after measurement” row. The post-measurement screen 651 is displayed on the display 340 at the time of transition to the post-measurement process, which is a partial change in the display contents of the initial setting screen 600.

The numerical value “210 μm²” of the specific area in the measured area display field 614 in FIG. 7 is only an example, and is not limited thereto. The measured area display field 614 indicates the actual area of the colonies 114 formed on the culture medium 113 of the measurement target as a result of the culturing of the biological entities 115 during the elapsed time when the mid-measurement process is executed. In the embodiment, the time when the measurement stop button 604 is operated is when one hour has elapsed after the operation of the measurement start button 603. That is, in the embodiment, the time length of the mid-measurement process is assumed to be one hour. Therefore, the elapsed time display field 611 in the post-measurement screen 651 indicates the time (01:00:00) one hour after the time (00:00:00) indicated by the elapsed time display field 611 of the initial setting screen 600.

The area of the “actual colonies” indicated by the measured area display field 614 is calculated by the area calculation function 421 (refer to FIG. 6).

Specifically, when the measurement start button 603 is operated, an image is captured by the detection device 100 in which the measurement target set. As a result, the raw data 3221 of the image (initial image) at a start time point of the mid-measurement process is output from the detection device 100 and transferred to the information processing device 300. When the measurement stop button 604 is operated, an image is captured by the detection device 100 in which the measurement target is set. As a result, the raw data 3221 of the image (final image) at the completion time point of the mid-measurement process is output from the detection device 100 and transferred to the information processing device 300. The raw data 3221 of the initial image and the final image is stored in the FTP directory 322, then converted by the area calculation function 421 into the pre-information-addition image data 3231, and stored in the first image directory 323. The area calculation function 421 calculates the area of the “actual colonies” indicated by the measured area display field 614 based on the size of the region of the biological entities 115 in the initial image and the size of the region occupied by the colonies 114 in the final image.

The culture medium 113 differs from the biological entities 115 in degree of light transmission. The culture medium 113 also differs from the colonies 114 in degree of light transmission. Therefore, regions of the biological entities 115 included in the initial image are identified by light/dark differences in the initial image, boundaries (edges) between the biological entities 115 and the culture medium 113 appeared by the light/dark differences, and other features. Regions of the colonies 114 included in the final image are identified by the light/dark differences in the final image, boundaries (edges) between the colonies 114 and the culture medium 113 appeared by the light/dark differences, and other features. The area calculation function 421 implements an image analysis processing algorithm to identify the regions of the biological entities 115 and the regions of the colonies 114 based on the light/dark differences and the edges in the image. Since such an image analysis processing algorithm may be known and is not particularly noted in the present disclosure, detailed description will not be made.

As described above, the area of the biological entities 115 is obtained as the “initial area”. Therefore, the area of a partial image that is considered to be the regions of the biological entities 115 in the initial image can be regarded as the “initial area”. For this reason, the “initial area” only needs to be multiplied by a ratio of the size of the partial image that is considered to be the region of colonies 114 in the final image to the size of the partial image that is considered to be the region of the biological entities 115 in the initial image. Such multiplication can calculate the area of the “actual colonies” indicated by the measured area display field 614. The area calculation function 421 implements an algorithm to calculate the area of the “actual colonies” indicated by the measured area display field 614 through this mechanism.

Thus, in the embodiment, the rate of increase of the actual colony area is calculated based on the ratio between the region of the biological entities (for example, Escherichia coli) included in the image represented by the output at a first time point and the region of the biological entities after the culturing included in the image represented by the output at a second time point. The first time point is a start time point of the culturing period of culturing the biological entities having the predetermined area (for example, 100 μm²) in the predetermined environment for the predetermined time (for example, one hour). The second time point is a completion time point of the culturing period. Herein, the start time point corresponds to a time point at which the measurement start button 603 included in the initial setting screen 600 is operated. The completion time point corresponds to a time point at which the measurement stop button 604 included in the initial setting screen 600 is operated.

The measurement result display field 615 is a field that indicates information on an error between the elapsed time displayed in the elapsed time display field 611 during the mid-measurement process and a calculated elapsed time. The “elapsed time displayed in the elapsed time display field 611 during the mid-measurement process” herein refers to the elapsed time (pre-measurement time) measured by the information processing device 300 using the system clock 500 until the measurement stop button 604 is operated. The “calculated elapsed time” refers to the elapsed time calculated based on the relation between the area indicated in the initial area setting field 602 and the area indicated in the measured area display field 614.

The calculation of the numerical value for the error indicated in the measurement result display field 615 is performed by the elapsed time calculation function 422 (refer to FIG. 6). First, the calculated elapsed time is obtained by Expression (1) below. In Expression (1), t on the left-hand side represents the calculated elapsed time. Aₜ on the right-hand side in Expression (1) represents the area indicated by the measured area display field 614, that is, the area of the “actual colonies” obtained after the completion of the mid-measurement process. A₀ represents the area indicated by the initial area setting field 602, that is, the initial area of the biological entities 115 before the mid-measurement process. A base (v) of a logarithm (log) is the “area increase rate” (refer to FIG. 8) of a type of bacteria applied (inoculated) onto the culture medium 113 as the biological entities to be cultured 115.

t=Logᵥ(Aₜ/A₀) (1)

The unit for “t” calculated by Expression (1) is hours. That is, if t=1, t is 1 hour. Substituting a numerical value corresponding to the post-measurement screen 651 in FIG. 7 into Expression (1) results in: t=log_{2.2}(210/100)=0.95. 0.95 hour is 57 minutes.

The processor 310, which executes the computer programs and other information stored in the storage 320 to serve as the elapsed time calculation function 422, calculates the calculated elapsed time (t) using Expression (1) explained above. The processor 310 subtracts the time length of the mid-measurement process displayed in the elapsed time display field 611 from the calculated elapsed time (t), thereby calculating the error between the calculated elapsed time (t) and the time length of the mid-measurement process using Expression (2) below. E on the left-hand side in Expression (2) indicates the error. t₀ on the right-hand side in Expression (2) represents the time length of the mid-measurement process displayed in the elapsed time display field 611. The unit for t₀ is hours.

E=t−t₀ (2)

If E is 0, it indicates that no error is present between the calculated elapsed time (t) and the elapsed time (pre-measurement time) measured by the information processing device 300 using the system clock 500 until the measurement stop button 604 is operated. If E is a positive value greater than 0, it indicates that the calculated elapsed time (t) is longer than the pre-measurement time. If E is a negative value below 0, it indicates that the calculated elapsed time (t) is shorter than the pre-measurement time.

The processor 310, which executes the computer programs and other information stored in the storage 320 to serve as the display control function 440, provides a display output illustrating the error (E) calculated by the function of the elapsed time calculation function 422, in the measurement result display field 615. A post-measurement screen 652 illustrated in the “display output contents related to measurement” column of the “after measurement” row in FIG. 8 is a screen after a display content “calculating” in the measurement result display field 615 on the post-measurement screen 651 has been switched to the display content indicating the error (E). In other words, until the calculation of the calculated elapsed time (t) and the error (E) is completed, the measurement result display field 615 has the display content “calculating” as illustrated in the post-measurement screen 651. On the post-measurement screen 652 in FIG. 7, the content of the measurement result display field 615 is “-0.05 hours (-3 minutes)”. This display content is only an example, and the display content is not limited thereto. Other display contents may be used as long as being capable of indicating the same meaning.

In the embodiment, the processor 310 serving as the display control function 440 applies a time correction corresponding to the error (E) to the time displayed in the elapsed time display field 611. Thus, the display content of the elapsed time display field 611 on the post-measurement screen 652 has changed from “01: 00:00” displayed in the elapsed time display field 611 on the post-measurement screen 651 to “00: 57:00” reflecting “−3 minutes” of the error (E).

The system clock setting function 430 in FIG. 6 adjusts the relation between time and clock in the system clock 500 to correspond to the calculated elapsed time (t), based on the calculated elapsed time (t) calculated by the elapsed time calculation function 422 and the error (E).

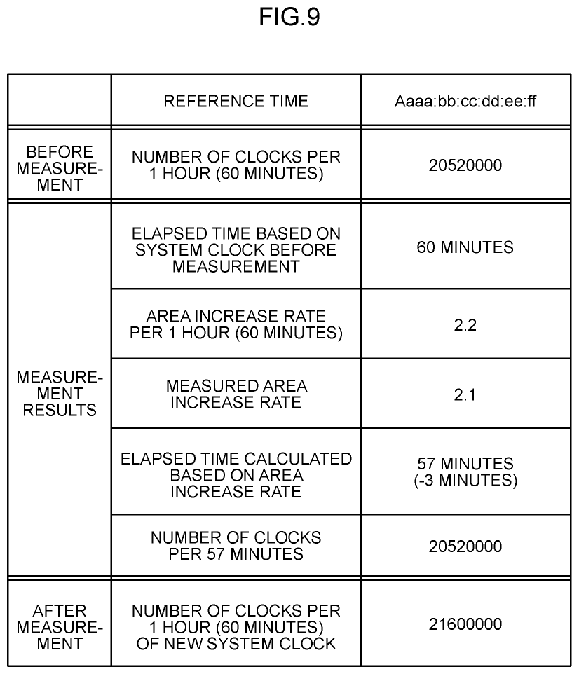

FIG. 9 is a table illustrating an example of the relation between time and clock before the measurement of the time measurement process, measurement results of the time measurement process, and the relation between time and clock after the measurement of the time measurement process.

The system clock 500 keeps time based on a reference time and the number of clocks per predetermined time. FIG. 9 illustrates “aaaa:bb:cc:dd:ee:ff” as the reference time. “aaaa” indicates the year (Christian year). “bb” indicates the month (any one of January to December). “cc” indicates the day (any one of the 1st to the 31st). “dd” indicates the hour (any one of 0 o'clock to 23 o'clock). “ee” indicates the minute (any one of 0 minutes to 59 minutes). “ff” indicates the second (any one of 0 seconds to 59 seconds).

FIG. 9 illustrates an example of the relation between time and clock before the measurement of the time measurement process where the “number of clocks per hour (60 minutes)” is “20520000” in the “before measurement” row. The “number of clocks” refers to the number of hardware clocks periodically generated by the real-time clock (RTC) circuit 501 provided in the information processing device 300. The expression that the “number of clocks per hour (60 minutes)” is “20520000” indicates that one hour elapses while the RTC circuit 501 generates 20520000 hardware clocks. In other words, the lapse of time can be managed according to the number of the hardware clocks generated by the RTC circuit 501. Time counting starts at the reference time. The reference time is, for example, Jan. 1, 1970, 00:00:00, but more recent year, month, day, minute, and second may be set as the reference time. The RTC circuit 501 is a known circuit and is, for example, a circuit for counting time using the same mechanism as a quartz clock that uses, as the hardware clocks, clock signals that can be generated using the inverse piezoelectric effect of a crystal oscillator (quartz).

Thus, the RTC circuit 501 of the information processing device 300 serves as the circuit that periodically generates the clocks for measuring the lapse of the predetermined time.

A “measurement results” row illustrated in FIG. 9 reflects the measurement results of the time measurement process illustrated in FIG. 7. Specifically, the value “60 minutes” that is the “elapsed time measured by the system clock before measurement” in the “measurement results” row means one hour, and indicates the display content of the elapsed time display field 611 on the post-measurement screen 651 described with reference to FIG. 7 and the length of time of the mid-measurement process. The value “2.2” that is the “area increase rate per hour (60 minutes)” in the “measurement results” row is the content of the area increase rate display field 613 described with reference to FIG. 7. The “measured area increase rate” in the “measurement result” row is the value of a multiplying factor corresponding to the ratio of the area of the “actual colonies” illustrated in the measured area display field 614 to the initial area of the biological entities 115 illustrated in the initial area setting field 602. That is, the “measured area increase rate” reflects the value “2.1” corresponding to the ratio of “210 μm²” illustrated in the measured area display field 614 to “100 μm²” illustrated in the initial area setting field 602. The “elapsed time calculated based on area increase rate” in the “measurement result” row is the calculated elapsed time (t) by Expression (1) given above.

The “number of clocks per 57 minutes” in the “measurement result” row illustrated in FIG. 9 is the number of the hardware clocks generated by the RTC circuit 501 during the calculated elapsed time (t). This number corresponds to the relation between the “number of clocks per hour (60 minutes)” in the “before measurement” row and the “elapsed time measured by the system clock before measurement” in the “measurement results” row. That is, since one hour (60 minutes) kept with the “number of clocks per hour (60 minutes)” in the “before measurement” row using the system clock 500 has elapsed in the mid-measurement process, the number of the hardware clocks generated during that time is 20520000. The calculated elapsed time (t) based on the mid-measurement process is “57 minutes”. Therefore, based on the calculated elapsed time (t), the number of the hardware clocks generated during the “57 minutes” is “20520000”.

The “number of clocks per hour (60 minutes) of new system clock” in the “after measurement” row illustrated in FIG. 9 is the number of the hardware clocks per hour. The number of the hardware clocks per hour is set according to the correspondence between the calculated elapsed time (t) and the number of the hardware clocks generated by the RTC circuit 501. Specifically, as indicated by the “number of clocks per 57 minutes” in the “measurement results” row, the number of the hardware clocks that occur in “57 minutes” is “20520000” based on the calculated elapsed time (t). Therefore, the number of the hardware clocks that occur during “60 minutes” based on the calculated elapsed time (t) is 20520000/57×60=21600000. Thus, the “number of clocks per hour (60 minutes) of new system clock” in the “after measurement” row is “21600000”.

As described above, based on the calculated elapsed time (t) calculated by the elapsed time calculation function 422 and the error (E), the system clock setting function 430 adjusts the relation between time and clock in the system clock 500 to correspond to the calculated elapsed time (t). Specifically, in the example illustrated in FIG. 9, the system clock setting function 430 sets the number of the hardware clocks per hour of the RTC circuit 501 from “20520000” before the time measurement process to “21600000” after the time measurement process. Consequently, the time to be kept by the system clock 500 after the time measurement process is set according to the correspondence between the calculated elapsed time (t) obtained in the time measurement process and the hardware clocks of the RTC circuit 501.

Thus, the processor 310 of the information processing device 300 executes the application programs 321 to serve as a setter. The setter newly set a relation between a predetermined time and the number of clocks (hardware clocks) to be generated by the RTC circuit 501 (the number of the clocks per the predetermined time), based on the relation between a first increase rate and a second increase rate. The first increase rate is the rate of increase of the actual colony area obtained based on the output indicating the result of culturing the biological entities (for example, Escherichia coli) with a predetermined area (for example, 100 μm²) in a predetermined environment for a predetermined time (for example, one hour) measured during the time measurement process based on the number of clocks generated by the RTC circuit 501. The second increase rate is the rate of increase indicated by the information included in the reference data 3211. For example, based on the relation between the first increase rate and the second increase rate, the setter sets the number of clocks of the RTC circuit 501 per the predetermined time from 20520000 to 21600000.

The numbers of the clocks “20520000” and “21600000” illustrated in FIG. 9 are only examples, and are not intended to limit the actual number of the hardware clocks to these numbers. The actual numbers of the clocks per hour (60 minutes) may be significantly greater than these examples.

The image analysis function 420 (refer to FIG. 6) of the embodiment performs a process of adding information indicating the calculated elapsed time (t), which is calculated by the elapsed time calculation function 422, to the image. The image analysis function 420 also performs a process of adding, to the image, information that reflects the time measured by the system clock 500 newly set by the system clock setting function 430 correspondingly to the calculated elapsed time (t). The data of the image to which such information has been added is stored in the second image directory 324 as the post-information-addition image data 3241.

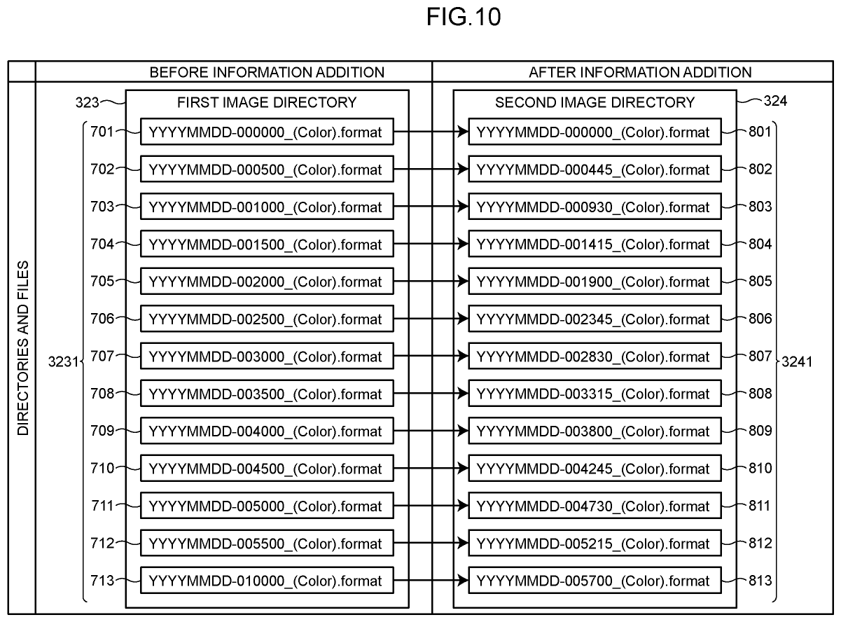

FIG. 10 is a diagram illustrating an example of relations between the directories and the files before and after the addition of information by the image analysis function 420. The example in FIG. 10 corresponds to the measurement results described with reference to FIGS. 7 and 9. The raw data 3221 transferred from the detection device 100 to the information processing device 300 is subjected to a format conversion process by the image format conversion processing function 410 before adding the information. The data after being subjected to the conversion process is stored in the first image directory 323 as the pre-information-addition image data 3231. The pre-information-addition image data 3231 illustrated in FIG. 10 includes 13 files, such as files 701, 702, . . . , 713, obtained for one hour that is the length of time of the mid-measurement process.

The naming rules of the files in the embodiment include adding a character string indicating the image acquisition time. For example, the name of the file 701 illustrated in FIG. 10 is “YYYYMMDD-000000_(Color). format”.

The character string “YYYYMMDD-000000” in the name is a character string indicating the acquisition time of the image saved as the file 701. “YYYY” indicates the year (Christian year). “MM” indicates the month (any one of January to December). “DD” indicates the day (any one of the 1st to the 31st). The six-digit number “000000” indicates that it is 0 o'clock, 0 minutes, 0 seconds. The first two of these six-digit numbers indicate the hour (any one of 0 o'clock to 23 o'clock), the next two digits indicate the minute (any one of 0 minutes to 59 minutes), and the next two digits indicate the second (any one of 0 seconds to 59 seconds).

The name of the file 702 is the same as the name of the file 701, except that “000000” in the name of the file 701 is replaced with “000500” indicating 0 o'clock, 5 minutes, 0 seconds. The name of the file 703 is the same as the name of the file 701, except that “000000” in the name of the file 701 is replaced with “001000” indicating 0 o'clock, 10 minutes, 0 seconds. Thereafter, based on the same concept, character strings indicating different times in increments of five minutes are included in the names of the files 704, 705, 706, 707, 708, 709, 710, 711, 712, and 713 in this order. Thus, in the embodiment, the images are captured by the detection device 100 and the files are transferred from the detection device 100 to the information processing device 300 at intervals of five minutes.

The character string “_(Color)” included in the name of the file indicates the color format of the file and can be changed as appropriate. For example, the character string “(Color)” may be replaced with “R” for a file obtained using red (R) light. The character string “(Color)” may be replaced with “G” for a file obtained using green (G) light. The character string “(Color)” may be replaced with “B” for a file obtained using blue (B) light. The “(Color)” string may be replaced by “RGB” for a file with a combination of the three RGB colors. The character string “. format” included in the name of the file is replaced with a character string of an extension corresponding to the predetermined image format employed in the process of the image format conversion processing function 410. Since PNG is employed in the embodiment, the character string of “. format” is, “. png”, for example.

As described above, the image analysis function 420 performs the process of adding, to the image, the information indicating the calculated elapsed time (t) calculated by the elapsed time calculation function 422. The image analysis function 420 also performs the process of adding, to the image, the information that reflects the time measured by the system clock 500 newly set by the system clock setting function 430 correspondingly to the calculated elapsed time (t). The files to which these information added is the files included in the pre-information-addition image data 3231. With the addition of such information, for example, files 801, 802, . . . , 813 are generated from the files 701, 702, . . . , 713 illustrated in FIG. 10, and stored in the second image directory 324.

As described with reference to FIG. 9, the number of clocks per 60 minutes before the time measurement process is treated as the number of clocks per 57 minutes after the execution of the time measurement process. Thus, the image analysis function 420 treats the elapsed time (60 minutes) of the mid-measurement process during which the files 701, 702, . . . , 713 have been acquired, as the time (57 minutes) kept by the system clock 500 after the execution. The image analysis function 420 gives, to the files 801, 802, . . . , 813, the character strings of the file names according to the time (57 minutes) kept by the system clock 500 after the execution.

For example, the name of the file 713 in the pre-information-addition image data 3231 is “YYYYMMDD-010000_(Color). format”. In contrast, the name of the file 813 in the second image directory 324 is “YYYYMMDD-005700_(Color). format”. This contrast reflects the fact that the time treated as one hour (60 minutes) before the measurement is treated as 57 minutes after the measurement. The name of file 702 in the pre-information-addition image data 3231 is

“YYYYMMDD-000500_(Color). format”. In contrast, the name of the file 802 in the second image directory 324 is “YYYYMMDD-000445_(Color). format”. This is due to the fact that, since the time 60 minutes is changed to be treated as 57 minutes from before to after the measurement, a lapse of 5 minutes is changed to be treated as a lapse of 4 minutes and 45 seconds. Based on the same concept, the files 803, 804, 805, 806, 807, 808, 809, 810, 811, and 812 are renamed from the files 703, 704, 705, 706, 707, 708, 709, 710, 711, and 712, respectively. As for the relation between the file 701 and the file 801, no difference is made in the file name. This is because the starting point of time is still treated the same even if the time 60 minutes is changed to be treated as 57 minutes from before to after the measurement.

In addition, in the embodiment, information on the culture medium 113 formed on the imaged placement substrate 111, information on the species of bacteria used as the biological entities to be cultured 115, and other information may be added to the file.

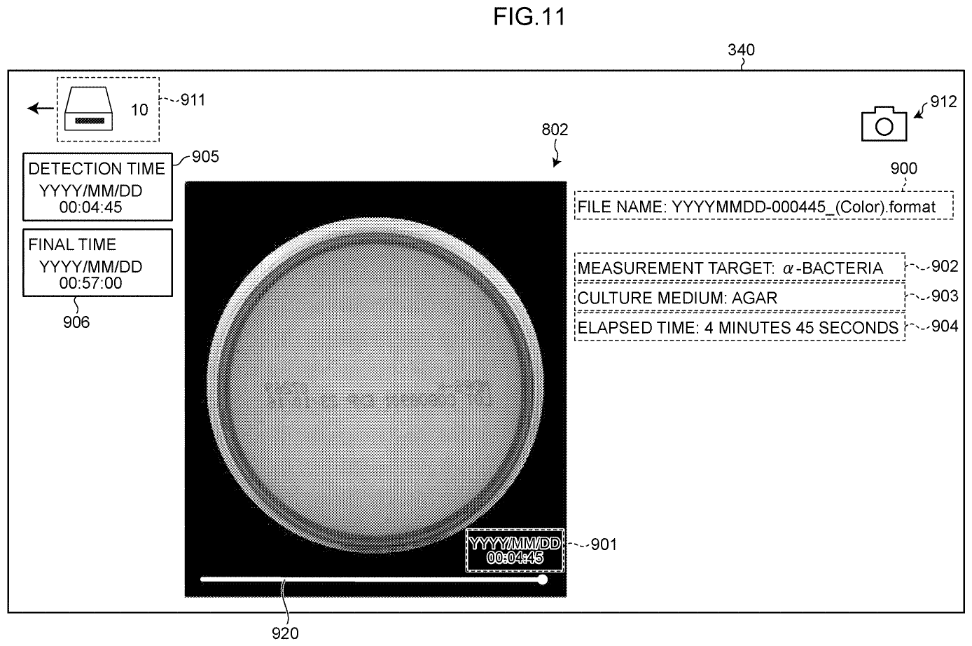

FIG. 11 is a diagram illustrating a display example of the file to which the information on the culture medium 113, the information on the species of bacteria used as the biological entities to be cultured 115, and other information are added. FIG. 11 illustrates the example where a file name 900, an acquisition time 901, bacterial species information 902, culture medium information 903, an elapsed time 904, an acquisition time 905, and a completion time 906 are further displayed when the file 802 is displayed.

The file name 900 indicates the name of the file being displayed. In FIG. 11, the file name 900 reflects the name of the file 802 because the file 802 is displayed.

The acquisition time 901 is a character string indicating the acquisition time of the file 802 that is embedded in the image of the file 802. Such a character string is obtained by combining a character string “YYYYMMDD” and a character string “000445” included in the name of the file 802.

The bacterial species information 902 indicates the bacterial species of the biological entities 115 applied (inoculated) onto the culture medium 113 in the object to be detected 110 set in the detection device 100 at the time of the acquisition of the file.

The culture medium information 903 indicates the information on the culture medium 113 in the object to be detected 110 set in the detection device 100 at the time of the acquisition of the file.

The elapsed time 904 indicates the time elapsed from the start time point of a series of processes (such as the time measurement process) for acquiring the file.

The acquisition time 905 is a character string similar to the acquisition time 901, but, unlike the acquisition time 901, is displayed outside a display field of the file 802.

The completion time 906 indicates the time of completion of the series of processes (such as the time measurement process) for acquiring the file.

The example illustrated in FIG. 11 further displays output source identification information 911, a snapshot button 912, and a slide bar 920. The output source identification information 911 indicates information to identify the detection device 100 from which the file was acquired. In the example illustrated in FIG. 11, a numerical value “10″ and a rough illustration of the detection device 100 are displayed side by side in the output source identification information 911. This illustration displays that the file was acquired by one of the detection devices 100 that is given the number ”10″.

The snapshot button 912 is a snapshot button for capturing the screen displayed on the display 340 at that point of time. The slide bar 920 is a slide bar for enlarging or reducing the displayed file.

In the embodiment, information for displaying the acquisition time 901, the bacterial species information 902, the culture medium information 903, the elapsed time 904, the acquisition time 905, the completion time 906, and the output source identification information 911 is added to the file as metadata of the file. The image analysis function 420 includes functions related to the addition of such information. The display control function 440 includes functions to achieve the display form on the display 340 illustrated in FIG. 11. The display form of the various types of information included in the metadata is not limited to the display form illustrated in FIG. 11, and can be changed as appropriate.

In the description with reference to FIGS. 9 to 11, the file management is performed based on the absolute time including year, month, and day, but the file management may be performed based on the elapsed time from the starting point of time, instead of the absolute time. For example, the file management may be performed based on the elapsed time from the starting time by regarding 0 hours, 0 minutes, and 0 seconds as the starting time of the operation of the detection device 100 in which the image is periodically acquired at intervals of five minutes. In this case, the characters “YYYYMMDD-” included in the file name in FIG. 10 are unnecessary. The first two digits of a six-digit character string such as “000000” may be a value exceeding 24. That is, a time equal to or greater than 24 hours may be expressed by six digits. If the operation is assumed to continue for 100 hours or longer, the six digits may be changed to seven or more digits.

In the example illustrated in FIG. 10, a file obtained by imaging the object to be detected 110 that is set for the purpose of newly setting the relation between the predetermined time and the number of clocks (for example, 21600000) of the RTC circuit 501 is subject to the processing of the image analysis function 420. However, the processing target of the image analysis function 420 is not limited to this file. For example, the placement substrate 111 on which the culture medium 113 with the biological entities 115 is formed is set in one or some (for example, one) of the detection devices 100. The bacterial species of the biological entities 115 are associated with information indicating the “initial area” and the “colony increase rate” in the reference data 3211 (refer to FIG. 6), such as the α-bacteria (for example, Escherichia coli) described above. Therefore, a “new relation between the predetermined time and the number of clocks (for example, 21600000) generated by the RTC circuit 501″ may be set based on the images represented by the outputs of the some of the detection devices 100. The new relation is based on the relation between the actual colony increase rate in the time measurement process and the colony increase rate indicated by the information included in the reference data 3211. When such setting is made, the new relation is reflected in the file name. In parallel with the time measurement process using the some of the detection devices 100, the placement substrate 111 is set in other (one or more) of the detection devices 100. The placement substrate 111 set in the other detection device(s) 100 is a placement substrate with the culture medium 113, onto which a strain of bacteria different from the biological entities 115 and having an unknown rate of increase is applied. The new relation may be reflected in files that reflect the images represented by the outputs of the other one or more of the detection devices 100. That is, additional information (for example, file name) may be given in the same way to files that reflect images represented by the outputs of the other detection devices 100 that produce outputs in parallel with the one of the detection devices 100. The additional information reflects the ”new relation between the predetermined time and the number of clocks (for example, 21600000) generated by the RTC circuit 501″ obtained based on the output of one of the detection devices 100. In this case, the processor 310 serving as the setter described above newly sets a relation between the predetermined time (for example, one hour) and the number of clocks generated by the circuit (RTC circuit 501) based on a certain output. The processor 310 serving as the setter provides the additional information, which corresponds to the newly set relation between the predetermined time and the number of generated clocks, to the outputs from some of the detection devices 100 and the other of the detection devices 100. The term “certain output” herein refers to the output obtained by culturing the biological entities (for example, Escherichia coli) having the predetermined area (for example, 100μm²) in the predetermined environment for the predetermined time (for example, one hour) measured based on the number of clocks generated by the circuit (RTC circuit 501), using the object to be detected (object to be detected 110) set in the some detection devices (some of the detection devices 100). In the additional information given herein, at least the character string of the six-digit numerical value serves as the “information indicating the time that is measured based on the newly set relation between the predetermined time and the number of clocks generated by the RTC circuit 501”. In the same concept, the various types of additional information described with reference to FIG. 11 may be individually given to the outputs from some of the detection devices 100 and the other of the detection devices 100. In this case, the character string as information indicating the time is written in the image represented by the output in the same way as in the case of the acquisition time 901, and also included the file name of the image and constitutes at least a part of the file name.

The operation of providing the additional information to the output of the other of the detection devices 100 need not be in parallel with the operation of providing the additional information to the output of the some of the detection devices 100 for setting the “new relation between the predetermined time and the number of clocks (for example, 21600000) generated by the RTC circuit 501”. Specifically, the time measurement process is first performed to set the “new relation between the predetermined time and the number of clocks (for example, 21600000) generated by the RTC circuit 501”, in the system clock 500. Additional information may be given to a file that reflects subsequently made progress of culturing of unknown bacterial species in the other of the detection devices 100. Such additional information is, for example, a file name indicating time based on the “new relation between the predetermined time and the number of clocks (for example, 21600000) generated by the RTC circuit 501” already set in the system clock 500.

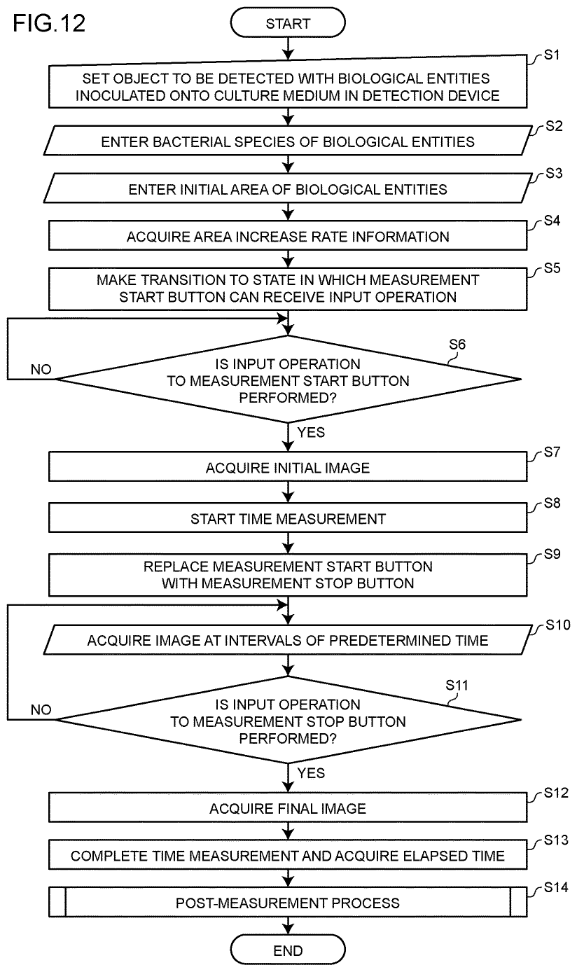

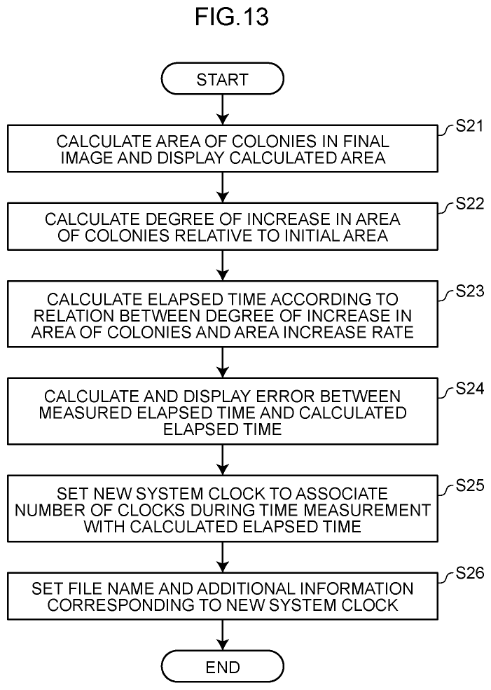

The following describes, in particular, the time measurement process in processing performed in the detection system 1 with reference to FIGS. 12 and 13. Unless otherwise noted, processes to be described with reference to FIGS. 12 and 13 are mainly performed by the processor 310 that reads out and executes the application programs 321.

FIG. 12 is a flowchart of the time measurement process. First, the object to be detected 110 with the culture medium 113 inoculated with the biological entities 115 is set in the detection device 100 (Step S1). Information indicating the bacterial species of the biological entities 115 is entered in the bacterial species setting field 601 of the initial setting screen 600 (refer to FIG. 7) (Step S2). The processes at Steps S1 and S2 are manually performed by a human, such as the administrator, in any order.

After the process at Step S2, the initial area of the biological entities 115 is set in the initial area setting field 602 (Step S3). The process at Step S3 may be performed by an input made by a human, such as the administrator, or the initial area may be the “initial area” that is associated by the reference data 3211 with the bacterial species entered in the bacterial species setting field 601 in the process at Step S2. The area increase rate obtained by referring to the reference data 3211 is set in the area increase rate display field 613 (Step S4). In the embodiment, along with the setting of the area increase rate display field 613, the assumed area display field 612 is also set. The information set in the assumed area display field 612 and the area increase rate display field 613 is the “area increase rate” and the “area after one hour” that are associated by the reference data 3211 with the bacterial species entered in the bacterial species setting field 601 in the process at Step S2.

After the processes at Step S1 to Step S4, the system transitions to a state in which the measurement start button 603 can receive an input operation (Step S5). In other words, in the embodiment, the measurement start button 603 on the initial setting screen 600 is inoperable until the contents of the bacterial species setting field 601, the initial area setting field 602, the assumed area display field 612, and the area increase rate display field 613 are filled.

After the process at Step S5 until the operation of the measurement start button 603 is performed, the processor 310 stays in a standby state in which the next process is not performed (No at Step S6). If the operation of the measurement start button 603 is performed (Yes at Step S6), at least the acquisition of the initial image is performed by the detection device 100 in which the object to be detected 110 has been set in the process at Step S1 (Step S7). The system clock 500 starts time measurement (Step S8). In the embodiment, the elapsed time measured by the process at Step S8 is displayed in the elapsed time display field 611 at any time. The measurement start button 603 is replaced with the measurement stop button 604 (Step S9).

After the process at Step S9, at least the acquisition of the image is performed by the detection device 100 in which the object to be detected 110 has been set in the process at Step S1, at intervals of a predetermined time (for example, five minutes) (Step S10). In the embodiment, the acquisition of the image is performed at intervals of five minutes, as described with reference to FIG. 10. The process at Step S10 continues until the operation of the measurement stop button 604 is performed (No at Step S11). After the process at Step S9, if the operation of the measurement stop button 604 is performed (Yes at Step S11), at least the acquisition of the final image is performed by the detection device 100 in which the object to be detected 110 has been set in the process at Step S1 (Step S12). The completion of the time measurement that has been started in the process at Step S8 and the acquisition of the elapsed time until the completion of the time measurement are performed (Step S13). After the processes at Steps S12 and S13, a post-measurement process is performed (Step S14).

FIG. 13 is a flowchart of the post-measurement process. First, the area of the colonies 114 in the final image acquired in the process at Step S12 is calculated, and the calculated area is displayed in the measured area display field 614 (Step S21). After the process at Step S21, the degree of increase (A_{t/}A₀) of the area of the colonies 114 calculated in the process at Step S21 relative to the initial area set at Step S3 is calculated (Step S22). After the process at Step S22, the elapsed time is calculated according to the relation between the degree of increase of the area (Aₜ/A₀) calculated in the process at Step S23 and the area increase rate set in the area increase rate display field 613 in the process at Step S4 (Step S23). Specifically, the calculated elapsed time (t) is calculated using Expression (1) given above in the process at Step S23.

After the process at Step S23, the elapsed time calculated in the process at Step S23 is displayed, and the error between the calculated elapsed time and the elapsed time until the completion of the time measurement obtained in the process at Step S12 is calculated and displayed (Step S24). The display by the process at Step S24 is performed in the measurement result display field 615.

After the process at Step S24, a new setting is made to the system clock 500 to associate the number of clocks during the elapsed time until the completion of the time measurement obtained in the process at Step S12 with the elapsed time calculated in the process at Step S23 (Step S25). After the process at Step S25, the name of a file (refer to FIG. 10) and the additional information (refer to FIG. 11) that correspond to the new setting of the system clock 500 are set (Step S26). With the completion of the process at Step S26, the post-measurement process that is the process at Step S14 illustrated in FIG. 12 is completed, and the time measurement process described with reference to FIG. 12 is completed.

As described above, according to the embodiment, the detection system 1 includes the detection device 100 and the information processing device 300 that is provided to be communicable with the detection device 100. The detection device 100 includes the planar optical sensor (planar optical sensor 81), the light source (light source 7) provided so as to be capable of emitting the light toward the surface for light detection by the planar optical sensor, and the object placement table (object placement table 4) that is interposed between the planar optical sensor and the light source and provided to allow the object to be detected (object to be detected 110) to be placed thereon. The object to be detected is configured to allow the culture medium (culture medium 113) to be formed thereon. The detection device 100 produces the output obtained by detecting the light from the light source transmitted through the object to be detected using the planar optical sensor. The information processing device 300 includes the storage (storage 320) that stores therein the reference data (reference data 3211) including the information indicating the rate of increase of the colony area (for example, 2.2) after the culturing with respect to the predetermined area that is assumed when the biological entities (for example, Escherichia coli) having the predetermined area (for example, 100 μm²) are cultured in the predetermined environment for the predetermined time (for example, one hour), the circuit (RTC circuit 501) that periodically generates the clocks for measuring the lapse of the predetermined time, and the setter (processor 310) that newly sets the relation between the predetermined time and the number of generated clocks (for example, 21600000) based on the relation between the rate of increase of the actual colony area obtained based on the output indicating the result of culturing of the biological entities having the predetermined area for the predetermined time measured during the time measurement process in the predetermined environment based on the number of clocks (for example, 20520000) and the rate of increase indicated by the information included in the reference data. This configuration allows the lapse of time to be managed based on the degree of expansion of the colonies exhibited by the biological entities. That is, the detection system can be operated under a time measurement rule conforming to the correspondence between the degree of expansion of the colonies and time assumed for the biological entities. Therefore, when monitoring microorganisms, biological tissues, or the like, for which the progress of changes, such as proliferation, is unknown, the progress of monitoring of such unknown changes can be managed based on the expansion pattern (time vs. area) of the colonies of the biological entities.

The information processing device 300 is provided independently from the detection device 100. This configuration allows the information processing device 300 to be provided in a different environment from the predetermined environment in which the detection device 100 is assumed to be provided, thus making the operation of the detection system 1 easier.

A plurality of the detection devices 100 are provided, and the setter (processor 310) sets the new relation between the predetermined time (for example, one hour) and the number of clocks generated by the circuit (RTC circuit 501) based on the predetermined output, and provides the additional information corresponding to the newly set relation between the predetermined time and the number of generated clocks to the outputs from some of the detection devices 100 and the other of the detection devices 100. The predetermined output is the output obtained by culturing the biological entities (for example, Escherichia coli) having the predetermined area (for example, 100 μm²) in the predetermined environment for the predetermined time measured based on the number of generated clocks, using the object to be detected (object to be detected 110) set in the some detection devices (some of the detection devices 100). This configuration allows the management of time based on the degree of expansion of the colonies exhibited by the biological entities to be applied also to the output related to the culturing of biological entities other than such biological entities.

The additional information (for example, file names and other information) includes the information indicating the time that is measured based on the newly set relation between the predetermined time (for example, one hour) and the number of clocks generated by the circuit (RTC circuit 501). Thus, a mechanism capable of more easily identifying the time measured based on the relation can be provided.

The character string as the information indicating time is written in the image represented by the output and constitutes at least a part of the file name of the image. Thus, a mechanism capable of more easily identifying the information indicating the time can be provided.

The information processing device 300 includes the display (display 340) that performs the display output corresponding to the processing content and the input device (input device 330) that receives the input to specify any one of the multiple types of biological entities (for example, Escherichia coli) .The display is configured to display the screen including the entry field (for example, bacterial species setting field 601) for specifying the biological entities. As a result, a mechanism can be provided that allows easier input to the information processing device 300 for the setting of the biological entities.

The setter (processor 310) calculates the rate of increase of the actual colony area based on the ratio between a first region and a second region. Herein, the first region is the region of the biological entities (for example, Escherichia coli) included in the image represented by the output at the start time point of the predetermined period. The predetermined period is the period of culturing the biological entities having the predetermined area (for example, 100 μm²) for the predetermined time (for example, one hour) in the predetermined environment. The second region is the region of the biological entities after being cultured included in the image represented by the output at the time of completion of the predetermined period. This configuration allows the calculation of the rate of increase of the actual colony area based on the clearer rule. Thus, the accuracy of the management of time based on the degree of expansion of the colonies exhibited by the biological entities can be ensured more easily.

The start time point of the period of culturing the biological entities (for example, Escherichia coli) having the predetermined area (for example, 100 μm²) in the predetermined environment for the predetermined time (for example, one hour) corresponds to the time point at which the measurement start button 603 included in the initial setting screen 600 is operated, and the time of completion of the period corresponds to the time point at which the measurement stop button 604 included in the initial setting screen 600 is operated. Thus, a mechanism can be provided that can establish a more intuitive correspondence between the timing of manual operations of the measurement start button 603 and the measurement stop button 604 and the start and completion of the period.

In the detection device 100 that communicates with the SNTP server 492 to obtain the information indicating the time, the control circuit 101 is assumed to manage the progression of time. Herein, the management of time by the control circuit 101 preferably synchronizes the management of time based on the system clock 500 of the information processing device 300. Specifically, as illustrated in FIG. 6, the SNTP server 492 preferably obtains the time measured by the system clock 500 and provides the information indicating the time to the detection device 100. In the embodiment, the images are captured by the detection device 100 and the files are transferred from the detection device 100 to the information processing device 300 on a regular basis, such as at intervals of five minutes. The operational cycle of the detection device 100 on such a regular basis also preferably synchronizes the system clock 500 managed by the information processing device 300. Specifically, the synchronization can be reliably achieved by providing the information indicating the time from the SNTP server 492 to the detection device 100 on a regular basis, such as at intervals of five minutes.

In the embodiment, the information processing device 300 is independent of the detection device 100, but hardware that functions in the same way as the information processing device 300 may be integrated with the detection device.

In the embodiment, the processor 310 serves as the setter through what is called software processing, but a dedicated hardware circuit that serves as the setter may be employed instead of the processor 310.

Other operational advantages accruing from the aspects described in the embodiment of the present disclosure that are obvious from the description herein, or that are conceivable as appropriate by those skilled in the art will naturally be understood as accruing from the present disclosure.

What is claimed is:

1. A detection system comprising:
a detection device; and
an information processing device that is provided so as to be communicable with the detection device, wherein the detection device comprises:
a planar optical sensor;
a light source; and
an object placement table,
the light source is provided so as to be capable of emitting light toward a detection surface of the planar optical sensor,
the object placement table is interposed between the planar optical sensor and the light source and provided to allow an object to be detected to be placed thereon,
the object to be detected is configured to allow a culture medium with biological entities to be formed thereon,
the detection device is configured to produce an output obtained by detecting the light from the light source transmitted through the object to be detected using the planar optical sensor, and
the information processing device comprises:
a storage configured to store reference data comprising information indicating a rate of increase of a colony area after culturing with respect to a predetermined area that is assumed when the biological entities having the predetermined area are cultured in a predetermined environment for a predetermined time;
a circuit configured to periodically generate clocks for measuring a lapse of the predetermined time; and
a setter configured to newly set a relation between the predetermined time and the number of the generated clocks based on a relation between the rate of increase of an actual colony area obtained based on the output indicating a result of culturing of the biological entities having the predetermined area for the predetermined time measured based on the number of clocks in the predetermined environment and the rate of increase indicated by the information.

2. The detection system according to claim 1, wherein the information processing device is provided independently from the detection device.

3. The detection system according to claim 1, wherein
a plurality of the detection devices are provided,
the setter is configured to:
newly set the relation between the predetermined time and the number of the generated clocks based on the output obtained by culturing the biological entities having the predetermined area for the predetermined time measured based on the number of clocks in the predetermined environment, using the object to be detected that is set in one or some of the detection devices; and
provide additional information corresponding to the newly set relation between the predetermined time and the number of the generated clocks to the outputs from the one or some of the detection devices and other of the detection devices.

4. The detection system according to claim 3, wherein the additional information comprises information indicating the time that is measured based on the newly set relation between the predetermined time and the number of the generated clocks.

5. The detection system according to claim 4, wherein a character string as the information indicating the time is written in an image represented by the output and constitutes at least a part of a file name of the image.

6. The detection system according to claim 1, wherein
the information processing device comprises
a display configured to perform display output corresponding to a processing content, and
an input device configured to receive input to specify any one of a plurality of types of the biological entities to be cultured, and
the display is configured to display a screen that includes an entry field to specify the biological entities to be cultured.

7. The detection system according to claim 6, wherein
the setter is configured to calculate the rate of increase of the actual colony area based on a ratio between:
a region of the biological entities included in the image represented by the output at a start time point of a period of culturing the biological entities having the predetermined area for the predetermined time measured based on the number of clocks in the predetermined environment, and
the region of the biological entities after the culturing included in the image represented by the output at a completion time point of the period.

8. The detection system according to claim 7, wherein
the start time point corresponds to a time point at which a measurement start button included in the screen is operated, and
the completion time point corresponds to a time point at which a measurement stop button included in the screen is operated.
